(19) United States
(12) Patent Application Publication
(10) Pub. No.: US 2003/0130224 A1
(43) Pub. Date: Jul. 10, 2003

(54) Expression of zeta negative and zeta positive nucleic acids using a dystrophin gene

(76) Inventors: Sean D. Monahan, Madison, WI (US); Jon A. Wolff, Madison, WI (US); Paul M. Slattum, Madison, WI (US); James E. Hagstrom, Madison, WI (US); Vladimir G. Budker, Madison, WI (US); David B. Rozema, Madison, WI (US)

Correspondence Address: Mark K. Johnson, Mirus Corporation, 505 S. Rosa Rd., Madison, WI, 53719, US

(21) Appl. No.: 10/339,934

(22) Filed: Jan. 10, 2003

Related U.S. Application Data
(63) Continuation-in-part of application No. 09/877,436, filed on Jun. 7, 2001.

(51) Int. Cl. A61K 48/00; A61F 5/00

(52) U.S. Cl. 514/44; 602/13

(57) ABSTRACT

Disclosed is expression of zeta negative and zeta positive nucleic acids or nucleic acid complexes using a dystrophin gene in a process for providing nucleic acid expression in a striated (skeletal or cardiac) muscle cell for the purpose of providing a change to the endogenous properties of the cell for cells affected by muscular dystrophy.

This Application is a Continuation-In-Part of application Ser. No. 09/877,436 filed June 1, 2001.

### FIELD OF THE INVENTION

The invention relates to treatment for various types of muscular dystrophy. More particularly, processes for the genetic repair or amelioration of the mutant phenotypes of dystrophic muscle cells are provided. The process provides for delivery of polynucleotides to tissue with a single injection.

### BACKGROUND

The muscular dystrophies (MD) are a heterogeneous group of mostly inherited disorders characterized by progressive muscle wasting and weakness which eventually leads to death. In most in not all forms of MD, the disease is associated with either a non-functioning or malfunctioning protein due to the presence of a mutant or deleted gene (Hartigan-O'Connor D and Chamberlain J S. Developments in Gene Therapy of Muscular Dystrophy. Microsc Res Tech 2000 48:223-238). Because of the nature of these diseases, few traditional treatments available. However, because the genes and protein products that are responsible for most of the dystrophies have been identified, delivery of corrective genes offers a promising treatment.

The challenge then is to repair the cellular genetic malfunction associated with a disease state, in this case muscular dystrophy, by delivery of a therapeutic exogenous polynucleotide to the cells. The polynucleotide must be delivered to a therapeutically significant percentage of a patient's muscle cells in a manner that is both efficient and safe. This polynucleotide, when delivered to a dystrophic cell, can compensate for a missing endogenous gene or block activity of a dominant negative endogenous gene. If genetic materials are appropriately delivered they can potentially enhance a patient's health and, in some instances, lead to a cure.

A significant obstacle to genetic repair of this disease is the large amount of post mitotic tissue that must be corrected. Fortunately, several attributes of striated muscle cells make genetic repair feasible. First, myofibers have a long life span, facilitating long term persistence of delivered genes. Second, for DMD and MCDM, its has been shown that gene replacement in striated muscle alone can alleviate the major features of the disease (Cox G A et al. Overexpression of dystrophin on transgenic mdx mice eliminates dystrophic symptoms without toxicity. Nature 1993 364:725-729; and Kuang W et al. Merosin-deficient congenital muscular dystrophy. Partial genetic correction in two mouse models [published erratum appears in J Clin Invest 1998 102(6):following 1275] J Clin Invest 1998 102:844-852). Third, dystrophin positive fibers may even possess a survival advantage over dystrophin negative fibers, suggesting that only a portion of the fibers need to receive the correcting polynucleotide (Morgan J E, Pagel C N et al. Long-term persistence and migration of myogenic cells injected into pre-irradiated muscles of mdx mice. J Neurol Sci 1993 115:191-200). Finally, only 20% of the normal level of dystrophin is required to be asymptomatic. Thus, low level dystrophin expression in a majority of muscle fibers may be sufficient for elimination of symptoms (Phelps S F et al. Expression of full length and truncated dystrophin mini-genes in transgenic mad mice. Hum Mol Genet 1995 4:1251-1258). Nevertheless, the target size remains very large.

Delivery of a nucleic acid means to transfer a nucleic acid from a container outside a mammal to near or within the outer cell membrane of a muscle cell in the mammal. The term transfection is used herein, in general, as a substitute for the term delivery, or, more specifically, the transfer of a nucleic acid from directly outside a cell membrane to within the cell membrane. If the nucleic acid is a DNA or cDNA, it enters the nucleus where it is transcribed into a messenger RNA that is then transported into the cytoplasm where it is translated into a protein. If the nucleic acid is an mRNA transcript, it is translated in the cytoplasm by a ribosome to produce a protein. If the nucleic acid is an anti-sense nucleic acid it can interfere with DNA or RNA function in either the nucleus or cytoplasm.

It was first observed that the in vivo injection of plasmid DNA into muscle enabled the expression of foreign genes in the muscle (Wolff, J A, Malone, R W, Williams, P, et al. Direct gene transfer into mouse muscle in vivo. Science 1990 247:1465-1468.). Since that report, several other studies have reported the ability for foreign gene expression following the direct injection of DNA into the parenchyma of other tissues. For example, naked DNA was expressed following its injection into cardiac muscle (Acsadi, G., Jiao, S., Jani, A., Duke, D., Williams, P., Chong, W., Wolff, J. A. Direct gene transfer and expression into rat heart in vivo. The New Biologist 1991 3(1), 71-81). However, since this method typically results in transfection of cells within only 1 cm of the injection site, direct parenchyma injections are impractical. Treatment of MD in this manner would require hundreds to thousands of injections to provide a therapeutic effect.

### SUMMARY

In a preferred embodiment, a process is described for the treatment of muscular dystrophy wherein a polynucleotide is delivered to a muscle cell, including skeletal and cardiac and muscle, of a mammal, comprising making a polynucleotide such as a nucleic acid, injecting the polynucleotide into a blood vessel, increasing the exit of the polynucleotide from vessels, and delivering the polynucleotide to a muscle cells within a tissue thereby altering endogenous properties of the cell. Increasing the permeability of the vessel consists of increasing the pressure within the vessel by rapidly injecting a large volume of fluid into the vessel and blocking the flow of blood into and out of the target tissue. This increased pressure is controlled by altering the injection volume of the solution, altering the rate of volume insertion, and by constricting the flow of blood into and out of the tissue during the procedure. The volume consists of a polynucleotide in a solution wherein the solution may contain a compound or compounds which may or may not complex with the polynucleotide and aid in delivery.

In a preferred embodiment, a complex for delivery of a polynucleotide to muscle cells is provided, comprising a complex consisting of a naked polynucleotide wherein the zeta potential, or surface charge, of the complex is negative. The polynucleotide codes either for a gene that expresses a therapeutic protein or a polynucleotide that can block function of a dominant deleterious endogenous gene. The complex is injected into a mammalian vessel and the permeability of the vessel is increased. Delivering the polynucleotide to the muscle cells thereby alters endogenous properties of the cells.

In a preferred embodiment, a complex for delivery of a polynucleotide to muscle cells is provided, comprising mixing a polynucleotide and a polymer(s) to form a complex wherein the zeta potential, or surface charge, of the complex is positive. The polymers may consist of polycations, polyanions, or both. The complex is injected into a mammalian vessel and the permeability of the vessel is increased. Delivering the polynucleotide to the muscle cells thereby alters endogenous properties of the cells.

In a preferred embodiment, a complex for delivery of a polynucleotide to muscle cells is provided, comprising mixing a polynucleotide and a polymer(s) to form a complex wherein the zeta potential, or surface charge, of the complex is not positive. The polymers may consist of polycations, polyanions, or both. The complex is injected into a mammalian vessel and the permeability of the vessel is increased. Delivering the polynucleotide to the muscle cells thereby alters endogenous properties of the cells.

In a preferred embodiment, a process is described for delivering a polynucleotide complexed with a compound into muscle cells, comprising making the polynucleotide-compound complex wherein the compound is selected from the group consisting of amphipathic molecules, polymers and non-viral vectors. The complex is injected into a mammalian vessel and the permeability of the vessel is increased. Delivering the polynucleotide to the muscle cells thereby alters endogenous properties of the cells.

In a preferred embodiment, a process is described for increasing the transit of the polynucleotide out of a vessel and into the muscle cells of the surrounding tissue, comprising rapidly injecting a large volume into a blood vessel supplying the target tissue, thus forcing fluid out of the vascular network into the extravascular space. This process is accomplished by forcing a volume containing a polynucleotide into a vessel and either constricting the flow of blood into and/or out of an area, adding a molecule that increases the permeability of a vessel, or both. The target tissue comprises the nonvascular parenchymal skeletal muscle cells supplied by the vessel distal to the point of injection and clamping. For injection into arteries, the target tissue is the muscles that the arteries supply with blood. For injection into veins, the target tissue is the muscles from which the veins drain the blood.

In a preferred embodiment, an in vivo process for delivering a polynucleotide to mammalian non-vascular muscle cells consists of inserting the polynucleotide into a blood vessel and applying pressure to the vessel proximal to the point of injection and target tissue. The process includes impeding blood flow by externally applying pressure to interior blood vessels such as by compressing mammalian skin. A device for applying pressure to mammalian skin for in vivo delivery of a polynucleotide to a mammalian cell is described. The device consists of a cuff, as defined in this specification, applied external to mammalian skin and around a limb to impede blood flow thereby increasing delivery efficiency of the polynucleotide to the mammalian cell. Compressing mammalian skin also includes applying a cuff over the skin, such as a sphygmomanometer or a tourniquet. However, it is important that the full function of the mammal's limbs be maintained subsequent to the delivery process. Full function means that the animal has equal or better use of the limb after the procedure compared to use of the limb prior to the procedure. The process especially consists of a polynucleotide delivered to non-vascular skeletal muscle cells.

In a preferred embodiment, an in vivo process for delivering a polynucleotide to mammalian non-vascular muscle cells consists of inserting the polynucleotide into a blood vessel and applying pressure to the vessel. The process includes constricting the flow of blood into and out of the target tissue by occluding blood flow through afferent and efferent vessels. Blood flow may be constricted by applying clamps directly to individual vessels, either externally or internally to the vessel itself.

In a preferred embodiment it may be preferential to immunosuppress the host receiving the nucleic acid. Immunosuppression can be of long term or short duration and can be accomplished by treatment with (combinations of) immunosuppresive drugs like cyclosporin A, ProGraf (FK506), corticosteroids, deoxyspergualin, and dexamethasone. Other methods include blocking of immune cell activation pathways, for instance by treatment with (or expression of) an antibody directed against CTLA4; redirection of activated immune cells by treatment with (or expression of) chemokines such as MIP-1a, MCP-1 and RANTES; and treatment with immunotoxins, such as a conjugate between anti-CD3 antibody and diphtheria toxin.

The defective genes that cause MD are known for many forms of the disease. These defective genes either fail to produce a protein product, produce a protein product that fails to function properly, or produce a dysfunctional protein product that interferes with the proper function of the cell. In a preferred embodiment, delivery of a polynucleotide encoding a therapeutically functional protein or a polynucleotide that inhibits production or activity of a dysfunctional protein is delivered to muscle cells of an MD patient for therapeutic treatment of the disease wherein the proteins that are expressed or inhibited by the polynucleotide are selected from the group that includes, but is not limited to: dystrophin (Duchene's and Becker MD); dystrophin-associated glycoproteins (β-sarcoglycan and δ-sarcoglycan, limb-girdle MD 2E and 2F; α-sarcoglycan and γ-sarcoglycan, limb-girdle MD 2D and 2C), calpain (autosomal recessive limb-girdle MD type 2A), caveolin-3 (autosomal-dominant limb-girdle MD), laminin-alpha2 (merosin-deficient congenital MD), fukutin (Fukuyama type congenital MD) and emerin (Emery-Dreifuss MD) or therapeutic variation of these proteins. In another preferred embodiment, a polynucleotide expressing a therapeutic protein beneficial to MD patients is delivered the muscle cells of the patient. These polynucleotides include, but are not limited to, those which encode and express: actin, titin, muscle creatine kinase, troponin, growth factors, human growth factor, vascular endothelial growth factor (VEGF), insulin, anti-inflammatory genes, etc.

The large size of some of genes involved in MD, for instance dystrophin, greatly limit their ability to be delivered by viruses. Viral delivery of genes to muscle cells is further hindered by the poor transport of viral vectors across normal vascular endothelium, even when high hydrostatic pressure is applied (Greelish J P et al. Stable restoration of the sarcoglycan complex in dystrophic muscle perfused with histamine and a recombinant adeno-associated viral vector. Nature Med. 1999 5:439-443; and Jejurikar S S et al. Induction of angiogenesis by lidocaine and basic fibroblast growth factor: a model for in vivo retroviral-mediated gene therapy. J Surg Res 1997 67:137-146). Increased pressure has only given better transduction of the vasculature itself, not the target muscle cells. Addition of papaverine and histamine along with adeno-associated virus did give more widespread, though delivery was still limited to muscle groups served by the perfused artery (Greelish J P, Su L T et al. Stable restoration of the sarcoglycan complex in dystrophic muscle perfused with histamine and a recombinant adeno-associated viral vector. Nature Med. 1999 5:439-443). Viral vectors are furthermore prone to generating an immune response which is recognized as one of the most important factors in limiting long term expression (Jooss K, Yang Y, Fisher K J, Wilson J M. Transduction of dendritic cells by DNA viral vectors directs the immune response to transgene products in muscle fibers. J Virol. 1998 May;72(5):4212-23).

Unlike viral delivery approaches, non-viral vectors (polynucleotides with or without associated compounds) are not limited in gene length capabilities, are much less immunogenic, and are readily and cheaply mass produced. These advantages allow for repeat injections which reduces an absolute requirement for very long term expression in transfected cells. We also offer a common development strategy for each type of MD, unlike viral delivery which must be optimized for each new gene.

Further objects, features, and advantages of the invention will be apparent from the following detailed description when taken in conjunction with the accompanying drawings.

### BRIEF DESCRIPTION OF FIGURES

FIG. 1. Muscle sections obtained 5 min (A and B) and 1 h (C) after 50 μg of Rh-pDNA in 10 ml of normal saline were injected within 7 sec into the femoral artery of rat either without impeding the outflow (A) or with impeding outflow (B and C). Arrows indicate Rh-pDNA between cells and arrowheads indicate pDNA inside myofibers. Magnification: 1260×.

FIG. 2 Expression of β-galactosidase (light grey) and GFP (white) in rat muscle injected intra-arterially at different times with the respective expression pDNAs. Panel A (640× magnification) is a low-power field illustrating that expression of β-galactosidase and GFP were typically not co-localized. Panels B and C are high power fields (1600× magnification) that show an example of co-localization (B) and separate expression (C)

FIG. 3. Photomicrographs of muscle sections histochemically stained for β-galactosidase expression. Panel A represents a muscle (pronator teres) with a high level of expression; panel B represents a muscle (abductor pollicis longus) with an average level of expression. Magnification: 160×.

FIG. 4. LacZ expression in mouse skeletal muscle seven days following intra-arterial injections of 100 μg pCI-LacZ (A) or pMI-DYS (B and C) in dystrophic mdx mouse (A and B) or normal ICR mouse (C)

FIG. 5. Illustration of luciferase expression in leg muscles of dystrophic and normal dog after intra-arterial injection of pCI-Luc plasmid under elevated pressure. Panel A shows expression distribution in normal dog. Panel B shows expression distribution in dystrophic dog model.

### DETAILED DESCRIPTION

We have found a method for delivering a therapeutic polynucleotide to striated muscle cells in a more even distribution than is possible with the current technology of direct intramuscular injections.

We describe a process for efficiently inserting a polynucleotide into mammalian striated muscle cells (skeletal muscle cells and cardiac muscle cells). More particularly, we have injected a polynucleotide expressing marker genes into limbs of rat, dog, and monkey and caused the polynucleotide to be delivered to and expressed in a large proportion of muscle cells throughout a limb. In addition, we have injected a polynucleotide expressing the dystrophin gene into limbs of both rat and dog MD models and caused the polynucleotide to expressed in a large proportion of muscle cells throughout a limb. For both the arm and leg injections, delivery was enhanced by using a cuff surrounding the arm or leg to impede blood flow away from the target tissue during the procedure. The expression levels achieved in monkeys indicate that the procedure is likely to be efficient in humans. It is noteworthy that expression levels in monkeys were similar to expression levels in rats since the efficiency of many prior art gene transfer techniques is less in larger animals.

A key advancement is the enhanced efficiency of polynucleotide delivery and expression in a larger distribution of cells that is achieved by increasing the extravasation of the polynucleotide from the tissue's blood vessels into the parenchyma (striated muscle tissue). Vessel permeability is increased by elevating blood pressure within the target tissue. The vessel pressure within the target tissue is increased by: delivering the injection fluid rapidly, using a large injection volume, constricting blood flow into and out of the tissue during the procedure, and/or increasing permeability of the vessel wall.

The term polynucleotide is a term of art that refers to a string of at least two nucleotides. Nucleotides are the monomeric units of nucleic acid polymers. Polynucleotides with less than 120 monomeric units are often called oligonucleotides. Natural polynucleotides have a ribose-phosphate backbone while artificial polynucleotides are polymerized in vitro and contain the same or similar bases but may contain other types of backbones. These backbones include: PNAs (peptide nucleic acids), phosphorothioates, phosphorodiamidates, morpholinos, and other variants of the phosphate backbone of native nucleic acids. The term includes deoxyribonucleic acid (DNA) and ribonucleic acid (RNA). DNA may be in form of cDNA, in vitro polymerized DNA, plasmid DNA, parts of a plasmid DNA, genetic material derived from a virus, linear DNA, chromosomal DNA, an oligonucleotide, anti-sense DNA, or derivatives of these groups. RNA may be in the form of tRNA (transfer RNA), snRNA (small nuclear RNA), rRNA (ribosomal RNA), mRNA (messenger RNA), anti-sense RNA, siRNA (small interfering RNA), RNAi, ribozymes, in vitro polymerized RNA, or derivatives of these groups. Anti-sense is a nucleic acid that interferes with the function of DNA and/or RNA. RNA interference (RNAi) describes the phenomenon whereby the presence of double-stranded RNA (dsRNA) of sequence that is identical or highly similar to a target gene results in the degradation of messenger RNA (mRNA) transcribed from that targeted gene (Sharp P A. RNA interference-2001. Genes Dev 2001 15:485-490). RNAi is likely mediated by siRNAs of approximately 21-25 nucleotides in length which are generated from the input dsRNAs (Hammond S M, Bernstein E, et al. An RNA-directed nuclease mediates post-transcriptional gene silencing in Drosophila cells. Nature 2000 404:293-296; Parrish S, Fleenor J, et al. Functional anatomy of a dsRNA trigger: differential requirement for the two trigger strands in RNA interference. Mol Cell 2000 6:1077-1087; Yang D, Lu H and Erickson J W. Evidence that processed small dsRNAs may mediate sequence-specific mRNA degradation during RNAi in Drosophila embryos. Curr Biol 2000 10:1191-1200; Zamore P D, Tuschl T, et al. RNAi: double-stranded RNA directs the ATP-dependent cleavage of mRNA at 21 to 23 nucleotide intervals. Cell 2000 101:25-33; Bernstein E, Caudy A A, et al. Role for a bidentate ribonuclease in the initiation step of RNA interference. Nature 2001 409:363-366). The polynucleotide can also be a sequence whose presence or expression in a cell alters the expression or function of endogenous genes or RNA. In addition these forms of DNA and RNA may be single, double, triple, or quadruple stranded.

A delivered polynucleotide can stay within the cytoplasm or nucleus apart from the endogenous genetic material. Alternatively, the polynucleotide could recombine with (become a part of) the endogenous genetic material. Recombination can cause the polynucleotide to be inserted into chromosomal polynucleotide by either homologous or non-homologous recombination.

A polynucleotide can be delivered to a cell to express an exogenous nucleotide sequence, to inhibit, eliminate, augment, or alter expression of an endogenous nucleotide sequence, or to express a specific physiological characteristic not naturally associated with the cell. Polynucleotides may be coded to express a whole or partial protein, or may be anti-sense.

An expression cassette refers to a natural or recombinantly produced polynucleotide that is capable of expressing protein(s). The cassette contains the coding region of the gene of interest and any other sequences that affect expression of the coding region. A DNA expression cassette typically includes a promoter (allowing transcription initiation), and a sequence encoding one or more proteins (transgene). Optionally, the expression cassette may include transcriptional enhancers, locus control regions, matrix attachment regions, scaffold attachment regions, non-coding sequences, splicing signals, transcription termination signals, and polyadenylation signals. An RNA expression cassette typically includes a translation initiation codon (allowing translation initiation), and a sequence encoding one or more proteins. Optionally, the expression cassette may include translation termination signals, a polyadenosine sequence, internal ribosome entry sites (IRES, Vagner S, Galy B, and Pyronnet S. Irresistible IRES. Attracting the translation machinery to internal ribosome entry sites. EMBO Rep 2001 October;2(10):893-898), and non-coding sequences.

Protein refers herein to a linear series of greater than 2 amino acid residues connected one to another as in a polypeptide. Proteins can be part of the cytoskeleton (e.g., actin, dystrophin, myosins, sarcoglycans, dystroglycans) and thus have a therapeutic effect in cardiomyopathies and musculoskeletal diseases (e.g., Duchene MD, limb-girdle MD).

The expression cassette promoter can be selected from any of the known promoters isolated from the group consisting of, but not limited to, the human genome, mammalian genomes, microbial genomes, viral genomes, and chimeric sequences. Additionally, artificially constructed sequences can be used that have shown to have promoter activity in the target cell type. Examples of viral promoters that have successfully been used to express transgenes include: human cytomegalovirus immediate early promoter, Rous sarcoma virus, Moloney leukemia virus, and SV40. Examples of mammalian promoters include: muscle creatine kinase, elongation factor 1, actin, desmin, and troponin. The choice of promoter in conjunction with other expression cassette elements can determine the level of transgene protein production in target cells. The expression cassette can be designed to express preferentially in muscle cell (operationally defined as a 5-fold higher expression level in the muscle cell compared to the average expression level in other cell types). A promoter, or combination of a promoter and other regulatory elements in the expression cassette, resulting in preferential expression in specific cell types is frequently referred to as tissue-specific. Preferential expression in muscle cells can be achieved by using promoters and regulatory elements from muscle-specific genes (e.g., muscle creatine kinase, myosin light chain, desmin, skeletal actin), or by combining transcriptional enhancers from muscle-specific genes with a promoter normally active in many cell types (e.g., the human cytomegalovirus immediate early promoter in combination with the myosin light chain enhancer). An example of a tissue-specific promoter is the muscle creatine kinase promoter, which expresses transgenes at high levels in skeletal muscle cells and at lower levels in other cell types (Johnson J E, Wold B J, Hauschka S D. Muscle creatine kinase sequence elements regulating skeletal and cardiac muscle expression in transgenic mice. Mol Cell Biol 1989 9(8):3393-9).

Alternatively, a DNA expression cassette may express an RNA sequence that is itself the active molecule, such as an anti-sense polynucleotide. Such anti-sense nucleic acids can block gene expression by preventing transcription of the gene or by preventing translation of a messenger RNA. Transcription can be blocked by the nucleic acid binding to the gene as a duplex or triplex. It could also block expression by binding to proteins that are involved in a particular cellular biochemical process. Ribozymes can also be used to destroy specific cellular RNA.

The polynucleotide may contain sequences that do not serve a therapeutic function in muscle cells but may be used in the generation of the polynucleotide. Such sequences include genes required for replication or selection of the polynucleotide in a host organism.

Parenchymal cells are the distinguishing cells of a gland or organ contained in and supported by the connective tissue framework. The parenchymal cells typically perform a function that is unique to the particular organ. The term “parenchymal” excludes cells that are common to many organs and tissues such as fibroblasts and endothelial cells within blood vessels.

Striated muscle includes skeletal and cardiac muscle and muscles of the diaphragm. Skeletal muscle cells include myoblasts, satellite cells, myotubules, and myofibers. Cardiac muscle cells include the myocardium, also known as cardiac muscle fibers or cardiac muscle cells, and the cells of the impulse connecting system such as those that constitute the sinoatrial node, atrioventricular node, and atrioventricular bundle. In a preferred embodiment skeletal muscle, cardiac muscle, or diaphragm muscle is targeted by injecting the polynucleotide into the blood vessel supplying the tissue.

Arterial or venous injection enables a polymer, oligonucleotide, polynucleotide, or polynucleotide containing complex to be delivered to more cells in a more even distribution than can be accomplished with direct intramuscular injections. Arterial and venous herein mean within the internal tubular structures called blood vessels that are connected to a tissue or organ within the body of an animal, including mammals, through which blood flows to or from a body part or tissue. Examples of vessels include arteries, arterioles, capillaries, venules, and veins.

Afferent blood vessels are defined as vessels in which blood flows toward the organ or tissue under normal physiological conditions. Efferent blood vessels are defined as vessels in which blood flows away from the organ or tissue under normal physiologic conditions. In the heart, afferent vessels are known as coronary arteries, while efferent vessels are referred to as coronary veins.

In a preferred embodiment, the permeability of the vessels within the target tissue is increased. Efficiency and distribution of polynucleotide delivery and expression is increased by increasing the permeability of the blood vessels within or near the target tissue. Permeability is defined here as the propensity for macromolecules such as polynucleotides or complexes of macromolecules to exit the vessel and enter the parenchyma (extravascular space). One measure of permeability is the rate at which macromolecules move through the vessel wall. Another measure of permeability is the lack of force that resists the movement of polynucleotides out of the vessel.

In a preferred embodiment, blood pressure and thus permeability within a tissue are increased by obstructing blood flow into and out of a target tissue and controlling the volume and rate of the injection of the polynucleotide containing fluid. For example, an afferent vessel supplying an organ is rapidly injected while the efferent vessel(s) (also called the venous outflow or tract) draining the tissue is ligated (partially or totally clamped) for a period of time sufficient to allow delivery of a polynucleotide. In the reverse, an efferent vessel is injected while an afferent vessel is transiently occluded.

In a preferred embodiment, a cuff is used to elevate blood pressure and therefore increase vessel permeability. The term cuff means a device for impeding blood flow through mammalian blood vessels. However, for purposes of the claims, cuff refers specifically to a device applied exterior to the mammal's skin and touches the skin in a non-invasive manner. In a preferred embodiment, the cuff is a device that applies external pressure around a mammalian limb and thereby pressure is applied internally to the blood vessel walls, thus constricting the flow of blood into and out of an organ or limb or other target tissue. Impeding blood flow causes blood pressure and thus vessel permeability to increase resulting in the blood and its contents (including the injected polynucleotides) to be urged out of the vessels and into the parenchyma. One example of a cuff is a sphygmomanometer which is normally used to measure pressure. Another example is a tourniquet.

In a preferred embodiment a polynucleotide or polynucleotide containing complex is arterially or venously injected in a large injection volume. The injection volume is dependent on the size of the animal to be injected and can be from 1.0 to 3.0 ml or greater for small animals (i.e. tail vein injections into mice). The injection volume for rats can be from 6 to 35 ml or greater. The injection volume for primates can be 70 to 200 ml or greater. The injection volume in terms of ml/body weight can be 0.03 ml/g to 0.1 ml/g or greater.

The injection volume can also be related to the target tissue. For example, delivery of a polynucleotide or polynucleotide complex to a limb can be aided by injecting a volume greater than 5 ml per rat limb or greater than 70 ml for a primate limb. The injection volumes in terms of ml/limb muscle are typically within the range of 0.6 to 1.8 ml/g of muscle but can be greater.

In another preferred embodiment the injection fluid is injected into a vessel rapidly. The speed of the injection is partially dependent on the volume to be injected, the size of the vessel into which the fluid is injected, and the size of the animal. In one embodiment the total injection volume (1-3 mls) can be injected in from 5 to 15 seconds into vessels of mice. In another embodiment the total injection volume (6-35 mls) can be injected into vessels of rats in from 7 to 20 seconds. In another embodiment the total injection volume (80-200 mls) can be injected into vessels of monkeys in 120 seconds or less.

In another preferred embodiment a large injection volume is used and the rate of injection is varied. Injection rates of less than 0.012 ml per gram (animal weight) per second are used in this embodiment. In another embodiment injection rates of less than 0.2 ml per gram (target tissue weight) per second are used for gene delivery to target organs. In another embodiment injection rates of less than 0.06 ml per gram (target tissue weight) per second are used for gene delivery into limb muscle and other muscles of primates.

In another preferred embodiment, the blood pressure within a vessel is increased by increasing the osmotic pressure within the blood vessel. Typically, hypertonic solutions containing salts such as NaCl, sugars or polyols such as mannitol are used. Hypertonic means that the osmolarity of the injection solution is greater than physiologic osmolarity. Isotonic means that the osmolarity of the injection solution is the same as the physiological osmolarity (the tonicity or osmotic pressure of the solution is similar to that of blood). Hypertonic solutions have increased tonicity and osmotic pressure relative to the osmotic pressure of blood and cause cells to shrink.

In another preferred embodiment, the permeability of the blood vessel can also be increased by a biologically-active molecule. A biologically-active molecule is a protein or a chemical such as papaverine or histamine that increases the permeability of the vessel by causing a change in function, activity, or shape of cells, such as the endothelial or smooth muscle cells, within the vessel wall. Typically, biologically-active molecules interact with a specific receptor, enzyme, or protein within the vessel cell to change the vessel's permeability. Such biologically-active molecules include vascular permeability factor (VPF) which is also known as vascular endothelial growth factor (VEGF). Another type of biologically-active molecule can increase permeability by changing the extracellular connective material. For example, an enzyme could digest the extracellular material and thereby increase the number and size of the holes of the connective material. Another type of biologically-active molecule is a chelator that binds calcium and thereby increases the endothelium permeability.

In yet another preferred embodiment the use of a cuff (or other external pressure device) is combined with the use of a pharmaceutical or biologically-active agent (such as papaverine) to increase vascular permeability.

In a preferred embodiment, the polynucleotide may be formed into a complex with another compound or compounds to enhance delivery. Such a compound can be a polymer such as a polycation or a polyanion.

A polymer is a molecule built up by repetitive bonding together of smaller units called monomers. In this application the term polymer includes short polymers which have two to 80 monomers (often called oligomers) and polymers having more than 80 monomers. The polymer can be linear, branched network, star, comb, or ladder types of polymer. The polymer can be a homopolymer in which a single monomer is used or can be copolymer in which two or more monomers are used. Types of copolymers include alternating, random, block and graft.

A polycation is a polymer containing a net positive charge, for example poly-L-lysine hydrobromide. The polycation can contain monomer units that are charge positive, charge neutral, or charge negative, however, the net charge of the polymer must be positive. A polycation also can mean a non-polymeric molecule that contains two or more positive charges. A polyanion is a polymer containing a net negative charge, for example polyglutamic acid. The polyanion can contain monomer units that are charge negative, charge neutral, or charge positive, however, the net charge on the polymer must be negative. A polyanion can also mean a non-polymeric molecule that contains two or more negative charges. The term polyion includes polycation, polyanion, zwitterionic polymers, and neutral polymers. The term zwitterionic refers to the product (salt) of the reaction between an acidic group and a basic group that are part of the same molecule. Salts are ionic compounds that dissociate into cations and anions when dissolved in solution. Salts increase the ionic strength of a solution, and consequently decrease interactions between nucleic acids with other cations.

One of our several methods of polynucleotide delivery to cells is the use of polynucleotide-polycation complexes. It was shown that cationic proteins like histones and protamines or synthetic polymers like polylysine, polyarginine, polyomithine, DEAE dextran, polybrene, and polyethylenimine are effective intracellular delivery agents.

In a preferred embodiment, polycations are mixed with polynucleotides for intra-arterial delivery to a muscle cells. Polycations provide the advantage of allowing attachment of polynucleotide to the target cell surface. The polymer forms a cross-bridge between the polyanionic nucleic acids and the polyanionic surfaces of the cells. As a result the main mechanism of polynucleotide translocation to the intracellular space might be non-specific adsorptive endocytosis which may be more effective then fluid phase endocytosis or receptor-mediated endocytosis. Furthermore, polycations are a convenient linker for attaching specific receptors to polynucleotide and as result, polynucleotide-polycation complexes can be targeted to specific cell types.

Additionally, polycations protect polynucleotides in complexes against nuclease degradation. This protection is important for both extra- and intracellular preservation of polynucleotide. The endocytic step in the intracellular uptake of polynucleotide-polycation complexes is suggested by results in which polynucleotide expression is only obtained by incorporating a mild hypertonic lysis step (either glycerol or DMSO). Gene expression is also enabled or increased by preventing endosome acidification with NH₄Cl or chloroquine. Polyethylenimine, which facilitates gene expression without additional treatments, probably disrupts endosomal function itself. Disruption of endosomal function has also been accomplished by linking the polycation to endosomal-disruptive agents such as fusion peptides, membrane active compounds, or viruses.

Polycations also cause DNA condensation. The Stokes radius (or effective volume) which one DNA molecule occupies in a complex with polycations is drastically lower than the Stokes radius of a free DNA molecule. The size of DNA/polymer complex may be important for gene delivery in vivo.

In a preferred embodiment, a condensed polynucleotide/polycation complex may be recharged (converting positive zeta potential to a less positive zeta potential or converting negative zeta potential to a less negative zeta potential) by addition of a polyanion to the complex. The resulting recharged complex can be formed with an appropriate amount of charge such that the resulting complex has a net negative, positive or neutral charge. The interaction between the polycation and the polyanion can be ionic, can involve the ionic interaction of the two polymer layers with shared cations, or can be crosslinked between cationic and anionic sites with a crosslinking system (including cleavable crosslinking systems, such as those containing disulfide bonds). The interaction between the charges located on the two polymer layers can be influenced with the use of added ions to the system. With the appropriate choice of ion, the layers can be made to disassociate from one another as the ion diffuses from the complex into the cell in which the concentration of the ion is low (use of an ion gradient). One of the advantages that flow from recharging DNA particles is reducing their non-specific interactions with cells and serum proteins (Wolff J et al. Hum Gene Therapy 1996 7:2123-2133; Dash et al. Gene Therapy 1999 6:643-650; Plank et al. Hum Gene Ther 1996 7:1437-1446; Ogris et al. Gene Therapy 1999 6:595-605; Schacht et al. Brit. Patent Application 9623051.1, 1996).

A wide a variety of polyanions can be used to recharge the DNA/polycation particles. They include (but not restricted to): Any water-soluble polyanion can be used for recharging purposes including succinylated PLL, succinylated PEI (branched), polyglutamic acid, polyaspartic acid, polyacrylic acid, polymethacrylic acid, polyethylacrylic acid, polypropylacrylic acid, polybutylacrylic acid, polymaleic acid, dextran sulfate, heparin, hyaluronic acid, polysulfates, polysulfonates, polyvinyl phosphoric acid, polyvinyl phosphonic acid, copolymers of polymaleic acid, polyhydroxybutyric acid, acidic polycarbohydrates, DNA, RNA, negatively charged proteins, pegylated derivatives of above polyanions, pegylated derivatives carrying specific ligands, block and graft copolymers of polyanions and any hydrophilic polymers (PEG, poly(vinylpyrrolidone), poly(acrylamide), etc).

Amphipathic, or amphiphilic, molecules have both hydrophilic (water-soluble) and hydrophobic (water-insoluble) parts. Hydrophilic groups indicate in qualitative terms that the chemical moiety is water-preferring. Typically, such chemical groups are water soluble, and are hydrogen bond donors or acceptors with water. Examples of hydrophilic groups include compounds with the following chemical moieties; carbohydrates, polyoxyethylene, peptides, oligonucleotides and groups containing amines, amides, alkoxy amides, carboxylic acids, sulfurs, or hydroxyls. Hydrophobic groups indicate in qualitative terms that the chemical moiety is water-avoiding. Typically, such chemical groups are not water soluble, and tend not to hydrogen bonds. Hydrocarbons are hydrophobic groups.

Polynucleotide or polymers may incorporate compounds that increase their utility. These groups can be incorporated into monomers prior to polymer formation or attached to the polymer after its formation. The group can be a protein, peptide, lipid, steroid, sugar, carbohydrate, nucleic acid or synthetic compound. The group may enhance cellular binding to receptors, cytoplasmic transport to the nucleus and nuclear entry or release from endosomes or other intracellular vesicles.

Nuclear localizing signals (NLS) enhance the targeting of the gene into proximity of the nucleus and/or its entry into the nucleus. Such nuclear transport signals can be a protein or a peptide such as the SV40 large T antigen NLS or the nucleoplasmin NLS. These nuclear localizing signals interact with a variety of nuclear transport factors which themselves could also function as NLS's since they are targeted to the nuclear pore and nucleus. Groups that enhance release from intracellular compartments (releasing signals) can cause polynucleotide release from intracellular compartments such as endosomes (early and late), lysosomes, phagosomes, vesicle, endoplasmic reticulum, Golgi, and sarcoplasmic reticulum. Release includes movement out of an intracellular compartment into cytoplasm or into an organelle such as the nucleus. Releasing signals include chemicals such as chloroquine, bafilomycin or Brefeldin A1, the ER-retaining signal (KDEL sequence), viral components such as influenza virus hemagglutinin subunit HA-2 peptides and other types of amphipathic peptides.

Cellular receptor signals are any signal that enhances the association of the gene with a cell. This increase in association can be accomplished by either increasing the binding of the gene to the cell surface and/or its association with an intracellular compartment, for example: ligands that enhance endocytosis by enhancing binding to the cell surface. This includes agents that target to the asialoglycoprotein receptor by using asialoglycoproteins or galactose residues. Other proteins such as insulin, EGF, or transferrin can be used for targeting. Peptides that include the RGD sequence can be used to target many cells. Chemical groups that react with sulfhydryl or disulfide groups on cells can also be used to target many types of cells. Folate and other vitamins can also be used for targeting. Other targeting groups include molecules that interact with membranes such as lipids fatty acids, cholesterol, dansyl compounds, and amphotericin derivatives. In addition viral proteins could be used to bind cells.

Reporter Molecules

There are four types of reporter (marker) gene products that are expressed from reporter genes. The reporter gene/protein systems include:

a) Intracellular gene products such as luciferase, β-galactosidase, or chloramphenicol acetyl transferase. Typically, they are enzymes whose enzymatic activity can be easily measured.

b) Intracellular gene products such as β-galactosidase or green fluorescent protein which identify cells expressing the reporter gene. On the basis of the intensity of cellular staining, these reporter gene products also yield qualitative information concerning the amount of foreign protein produced per cell.

c) Secreted gene products such as growth hormone, factor IX, or alpha1-antitrypsin are useful for determining the amount of a secreted protein that a gene transfer procedure can produce. The reporter gene product can be assayed in a small amount of blood.

d) Anti-sense polynucleotides which reduce expression from a known gene, which can then be measured.

We have disclosed gene expression achieved from reporter genes in parenchymal cells. The terms “delivery,” “delivering genetic information,” “therapeutic” and “therapeutic results” are defined in this application as representing levels of genetic products, including reporter (marker) gene products, which indicate a reasonable expectation of genetic expression using similar compounds (nucleic acids), at levels considered sufficient by a person having ordinary skill in the art of delivery and gene therapy. For example: Hemophilia A and B are caused by deficiencies of the X-linked clotting factors VIII and IX, respectively. Their clinical course is greatly influenced by the percentage of normal serum levels of factor VIII or IX: <2%, severe; 2-5%, moderate; and 5-30% mild. This indicates that in severe patients only 2% of the normal level can be considered therapeutic. Levels greater than 6% prevent spontaneous bleeds but not those secondary to surgery or injury. A person having ordinary skill in the art of gene therapy would reasonably anticipate therapeutic levels of expression of a gene specific for a disease based upon sufficient levels of marker gene results. In the Hemophilia example, if marker genes were expressed to yield a protein at a level comparable in volume to 2% of the normal level of factor VIII, it can be reasonably expected that the gene coding for factor VIII would also be expressed at similar levels.

### EXAMPLES

The high luciferase and β-galactosidase levels achieved in monkeys indicate that the procedure is likely to be efficient in humans. Expression levels were somewhat higher in monkeys than in rats.

The intra-arterial procedure requires that blood flow be impeded for substantially less than the time required for tissue damage caused by ischemia. In fact, a common anesthesia for human limb surgery (e.g., carpal tunnel repair) involves the blockage of blood flow for over one hour. We have not observed any widespread histological evidence of ischemic muscle damage in rats, dogs, or primates following the injections. The minimal elevations of muscle-derived enzymes discovered in the serum provide significant evidence against any consequential muscle damage.

The elevated blood pressure is potentially damaging to the arteries. However, we have observed minimal intimal changes in the arteries these are presumed to be transient and without consequence. Nonetheless, this minimal arterial damage may be prevented by better controlling the pressure.

For this pDNA administration procedure, several factors limit expression to the non-target tissue. 1) The tourniquet cuff, or other vessel clamping prevents the immediate spread of vector outside of the limb. 2) Efficient pDNA expression in the non-vascular parenchymal cells requires extravasation of the injected pDNA which only occurs in the target tissue.

The procedure requires relatively large amounts of pDNA to be administered. This amount of pDNA has not been associated with any toxic effects in rodents, dogs, or monkeys. Given that the tourniquet delays pDNA distribution outside of the limb and that intravascular pDNA is rapidly degraded by circulating DNases, pDNA toxicity is unlikely. In addition, the cost for producing clinical grade pDNA is considerably less expensive than viral vectors and does not represent an obstacle to its clinical use.

1. Polynucleotide Delivery to Limb Skeletal Muscle Cells in Rats

A. Delivery of polynucleotide to multiple skeletal muscles in rat via a single Injection into a blood vessel: 500 μg of pCI-Luc in 10 ml of normal saline solution was injected into the femoral artery of adult rats in which a tourniquet was applied to the outside of the leg proximal (tourniquet was applied to the upper portion of the quadriceps group of muscles) to the injection site. Five days after injection, the different muscle groups from the leg were removed and cut into equal sections. Each section was placed into lysis buffer, the muscles were homogenized and 10 μl of the resulting lysates were assayed for luciferase activity.

High levels of luciferase expression were expressed in all muscle groups that were located distal to the tourniquet. These included the biceps femoris, posterior muscles of the upper leg, gastrocnemius, muscles of the lower leg, and muscles of the plantar surface.

Table 1: Luciferase expression in the various muscles of the rat leg after the injection of 500 μg of pCI-Luc into the femoral artery with a tourniquet applied around the outside of the upper leg muscles.

Intravascular Delivery to Rat Leg (±External Tourniquet)

| | +Tourniquet | −Tourniquet |
|---|---|---|
| | Total Luciferase | Total Luciferase |
| Muscle Group | (ng/muscle group) | (ng/muscle group) |
| Upper leg anterior | 0.181* | 0.010 |
| (quadriceps) | | |
| Upper leg middle | 28.3 | 0.011 |
| (biceps femoris) | | |
| Upper leg posterior | 146.0 | 2.16 |
| (hamstrings) | | |
| Lower leg posterior | 253.6 | 1.57 |
| (gastrocnemius) | | |
| Lower leg anterior | 115.2 | 0.72 |
| (lower shin muscles) | | |
| Muscles of the plantar surface | 0.433 | 0.202 |

*majority of this muscle group was above the tourniquet

Expression of intra-arterially administered plasmid DNA was significantly enhanced in multiple muscle groups when blood flow was impeded using an external tourniquet.

B. Delivery of PEI/DNA and histone H1/DNA particles to multiple skeletal muscles in rat via a single injection into a blood vessel: PEI/DNA and histone H1/DNA particles were injected into rat leg muscle by a single intra-arterial injection into the external iliac (Budker et al. Gene Therapy 1998 5:272). Female Harlan Sprague Dawley (HSD SD) rats, approximately 150 g, were used for the muscle injections. Each received complexes containing 100 μg plasmid DNA encoding the luciferase gene under control of the CMV enhancer/promoter (pCI-Luc) (Zhang et al. Hum Gene Therapy 1997 8:1763).

Results of the rat injections are provided in relative light units (RLUs) and micrograms (lag) of luciferase produced. To determine RLUs, 10 μl of cell lysate were assayed luminometer and total muscle RLUs were determined by multiplying by the appropriate dilution factor. To determine the total amount of luciferase expressed per muscle we used a conversion equation that was determined in an earlier study (Zhang et al. Hum Gene Therapy 1997 8:1763) [pg luciferase=RLUs×5.1×10⁻⁵].

TABLE 2
Luciferase expression in multiple muscles of the leg
following injection of negatively charged
DNA/PEI or DNA/Histone HI particles.

| | Total | Total | |
|---|---|---|---|
| Muscle Group | RLUs | Luciferase | |
| DNA/PEI particles (1:0.5 charge ratio) | | | |
| muscle group 1 (upper leg anterior) | 3.50 × 10⁹ | 0.180 | μg |
| muscle group 2 (upper leg posterior) | 3.96 × 10⁹ | 0.202 | μg |
| muscle group 3 (upper leg medial) | 7.20 × 10⁹ | 0.368 | μg |
| muscle group 4 (lower leg posterior) | 9.90 × 10⁹ | 0.505 | μg |
| muscle group 5 (lower leg anterior) | 9.47 × 10⁸ | 0.048 | μg |
| muscle group 6 (foot) | 6.72 × 10⁶ | 0.0003 | μg |
| DNA/histone H1 particles | | | |
| (1:0.5 charge ratio) | | | |
| muscle group 1 (upper leg anterior) | 3.12 × 10⁹ | 0.180 | μg |
| muscle group 2 (upper leg posterior) | 9.13 × 10⁹ | 0.202 | μg |
| muscle group 3 (upper leg medial) | 1.23 × 10¹⁰ | 0.368 | μg |
| muscle group 4 (lower leg posterior) | 5.73 × 10⁹ | 0.505 | μg |
| muscle group 5 (lower leg anterior) | 4.81 × 10⁸ | 0.048 | μg |
| muscle group 6 (foot) | 6.49 × 10⁶ | 0.0003 | μg |

Results indicated high level luciferase expression throughout the leg with a single injection of DNA/PEI or DNA/histone H1 negative zeta potential complexes.

C. Injection of plasmid DNA (pCI-Luc)/M66 complexes into the iliac artery of rats:

Complex formation—500 μg pDNA (500 μl) was mixed with M66 copolymer at a 1:3 wt:wt ratio in 500 μl saline. Complexes were then diluted in Ringers solution to total volume of 10 mls.

Injections—a total volume of 10 mls was injected into the iliac artery of Sprague-Dawley rats (Harlan, Indianapolis, Ind.) in approximately 10 seconds.

Expression—Animals were sacrificed after 1 week and individual muscle groups were removed and assayed for luciferase expression.

TABLE 3
Luciferase expression in multiple muscles
of the leg following injection of DNA/M66 particles.

| | Total | Total |
|---|---|---|
| Muscle Group | RLUs | Luciferase |
| muscle group 1 (upper leg anterior) | 3.58 × 10⁹ | 0.183 μg |
| muscle group 2 (upper leg posterior) | 6.46 × 10⁸ | 0.032 μg |
| muscle group 3 (upper leg medial) | 2.63 × 10⁹ | 0.134 μg |
| muscle group 4 (lower leg posterior) | 1.97 × 10⁹ | 0.101 μg |
| muscle group 5 (lower leg anterior) | 3.19 × 10⁹ | 0.163 μg |

These results indicate that high level gene expression muscle groups of the leg was facilitated by delivery of pCI-Luc/M66 complexes into rat iliac artery.

D. Enhancement by M-methyl-L-arzinine (L-NMMA) of in vivo gene expression following intravascular delivery of a complex with negative zeta potential consisting of naked polynucleotide: Intravascular delivery of pCI-Luc via the iliac artery of rat following a short pre-treatment with L-NMMA delivery enhancer. A 4 cm long abdominal midline excision was performed in 150-200 g, adult Sprague-Dawley rats anesthetized with 80 mg/mg ketamine and 40 mg/kg xylazine. Microvessel clips were placed on external iliac, caudal epigastric, internal iliac and deferent duct arteries and veins to block both outflow and inflow of the blood to the leg. 3 ml of normal saline with 0.66 mM L-NMMA were injected into the external iliac artery. After 2 min, a 27 g butterfly needle was inserted into the external iliac artery and 10 ml of DNA solution (50 μg/ml pCI-Luc) in normal saline was injected within 8-9 sec. Luciferase assays were performed on limb muscle samples (quadriceps femoris) 2 days after injection. Luciferase expression was determined as previously reported (Wolff J A, Malone R W, et al. Direct gene transfer into mouse muscle in vivo. Science 1990 247:1465-1458) A LUMAT™ LB 9507 (EG&G Berthold, Bad-Wildbad, Germany) luminometer was used.

TABLE 4
Luciferase expression in rat leg muscle
following injection of DNA into the iliac artery.
Enhancement of delivery with M-methyl-L-arginine.

| | | Total |
|---|---|---|
| Organ | Treatment | Luciferase (nanograms) |
| Muscle (quadriceps) | +papaverine | 9,999 |
| Muscle (quadriceps) | +0.66 mM L-NMMA | 15,398 |
| Muscle (quadriceps) | +papaverine, +0.66 mM | 24,829 |
| | L-NMMA | |

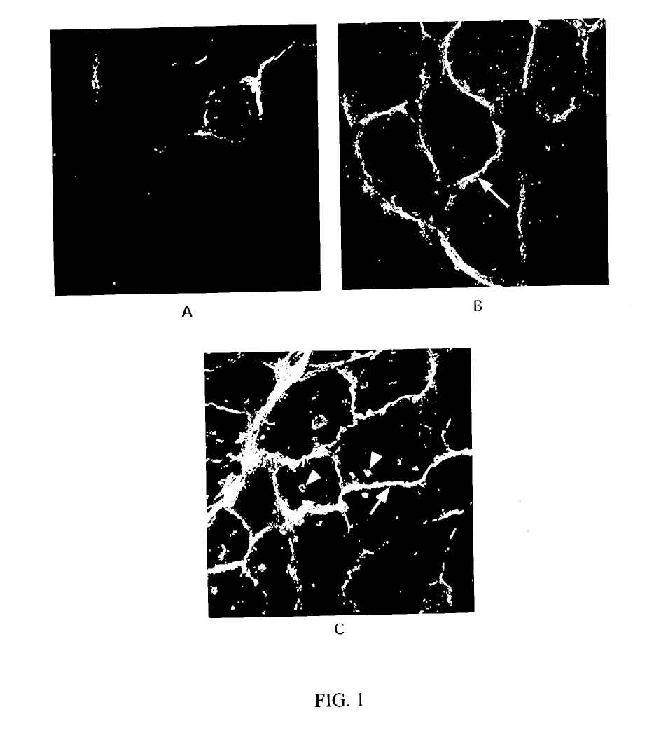

E. Labeled pDNA Distribution in Muscle: Rhodamine-labeled pDNA (Rh-pDNA) was injected into the femoral artery of rats under various conditions in order to explore the uptake mechanism in muscle. When the injections were performed without impeding blood outflow (low blood pressure), almost no DNA was detected within the muscle tissues or vessels. FIG. 1A presents a rare field when some DNA can be seen between muscle cells. When the injections were performed with outflow occlusion (increased blood pressure), Rh-pDNA was detected throughout all the muscle (FIGS. 1B and C). At 5 min after injection, examination of tissue sections indicated that the majority of the Rh-pDNA was surrounding the muscle cells and there was no intracellular staining (FIG. 1B). At one hour after injection, substantial amounts of DNA can be seen inside the cells (FIG. 1C). Examination of serial confocal sections indicates that the intracellular staining pattern is punctate, unlikely consistent with a T tubular distribution.

F. Timecourse of Muscle Expression After Intravascular Injection in Rats: Muscle luciferase expression was measured at several time points following delivery of the luciferase gene under control of either the CMV promoter (pCI-Luc⁺) or MCK promoter (pMI-Luc⁺) into: a) untreated rats, b) transiently immunosuppressed (treated with 10 mg/kg of FK506 orally and 1 mg/kg dexamethasone subcutaneously one day prior to, one hour prior to and one day after intra-arterial delivery of pDNA) or c) rats continuously immunosuppressed (treated with 2.5 mg/kg of FK506 orally and 1 mg/kg dexamethasone subcutaneously one day prior to, one hour prior to and every day thereafter with FK506) (Table 5). In untreated rats, luciferase expression was lost after 7 days from the CMV promoter or after 21 days from the MCK promoter. In either pCI-Luc⁺ or pMI-Luc⁺ injected rats, anti-luciferase antibodies were detected using ELISA by day 21 and were present at higher levels at day 56 and 70 after pDNA delivery (data not shown).

TABLE 5
Time course of luciferase expression (ng/g muscle)
in hindlimbs following intra-arterial injections with
500 μg of pCI-Luc+ (A) or pMI-Luc+ (B)
into rats treated with various immunosuppression regimens.

| | CONDITION | | |
|---|---|---|---|
| | | Transient | Continuous |
| Time After | No | Immuno- | Immuno- |
| Injection (Days) | Treatment | suppression | suppression |
| A. pCI-Luc+ | | | |
| 2 | 990.9 | | |
| 7 | 492.6 | | |
| 21 | 22.1 | | |
| 30 | 10.3 | 672.0 | 1212.0 |
| 56 | 0.3 | | |
| 70 | 0.1 | 17.3 | 464.0 |
| B. pMI-Luc+ | | | |
| 2 | 37.3 | | |
| 7 | 499.9 | | |
| 21 | 286.9 | | |
| 30 | | 1260.0 | |
| 56 | 3.3 | | |
| 70 | 0.3 | 571.0 | 1140.0 |

G. Expression Following Repetitive Injections: Sprague-Dawley rats (150 g) were injected intra-arterially in the right leg using 500 μg of pCI-Luc⁺ under increased pressure conditions on day 0. On days 7 and 14 the rats were injected slowly with 300 μg pCI-Luc⁺ in 1 ml into the tail vein. On day 24, the left leg was injected intra-arterially with 500 μg of pCI-Luc⁺. On day 26, the animals were sacrificed and the left leg revealed luciferase expression (mean=4,500 ng of total luciferase/leg muscles, n=2) similar to the levels achieved in animals not pre-injected with pDNA (mean=6,940 ng/leg muscles, n=26);

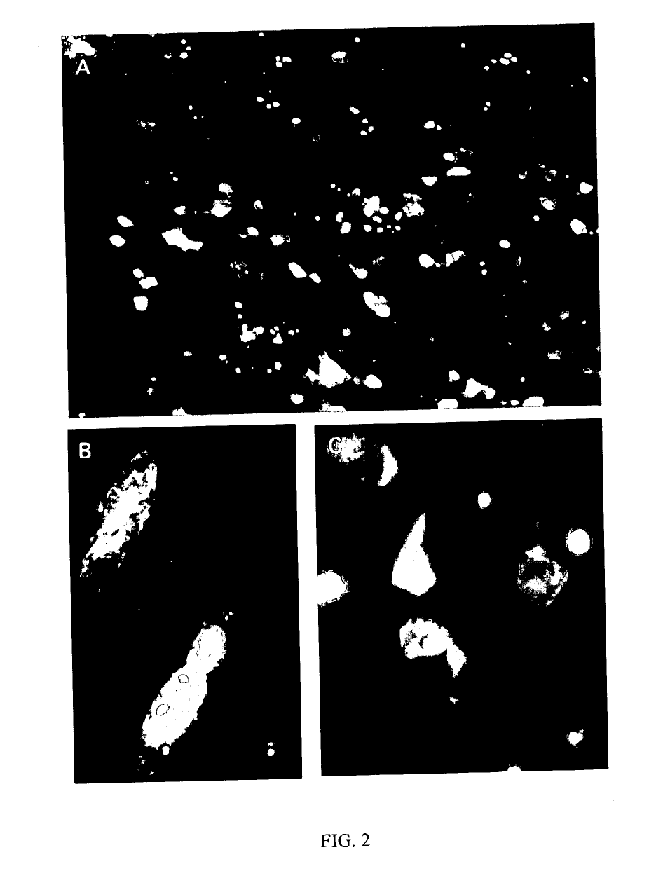

H. Repetitive Injections to Target a Larger Percentage of Myofibers: In order to explore the ability to access different populations of myofibers, the same leg in rats were injected with the 500 μg of the β-galactosidase plasmid (pCI-LacZ) and two days later with 500 μg of the nuclear GFP plasmid (pEBFP-N1). At two days after the last injection, the muscles were analyzed for expression of the two reporter genes. Expression of GFP and β-galactosidase was most often located in different myofibers (FIGS. 2A and C), but in some cells expression was coincident (FIG. 2B).

I. Delivery of Negatively vs. Positively Charged Complexes into Skeletal Muscle: Delivery of DNA/polycation particles into rat skeletal muscle via injection into an artery. These experiments were carried out to compare the delivery efficiency of negatively charged DNA complexes vs. positively charged DNA complexes into mammalian skeletal muscle.

All particles were injected into rat leg muscle via a single intra-arterial injection into the external iliac (Budker V et al. Gene Therapy 1998 5:272). Harlan Sprague Dawley (HSD SD) rats were used for the muscle injections. All rats used were female and approximately 150 g and each received complexes containing 100 μg of plasmid DNA encoding the luciferase gene under control of the CMV enhancer/promoter (pCI-Luc) (Zhang et al. 1997 Human Gene Therapy, 8:1763).

Results of the rat injections are in relative light units (RLUs) and μg of luciferase produced. To determine RLUs, 10 μl of cell lysate were assayed using a EG&G Berthold LB9507 luminometer and total muscle RLUs were determined by multiplying by the appropriate dilution factor. To determine the total amount of luciferase expressed per muscle we used a conversion equation that was determined in an earlier study (Zhang et al. Hum Gene Therapy 1997 8:1763) [ng luciferase=RLUs×5.1×10^{-8].}

Results: To achieve efficient gene delivery to cells growing in culture (in vitro) using DNA/polycation complexes, an excess of the polycation is needed relative to the DNA [(+) to (−) charge ratio >1]. When the polycation is in excess over the DNA, the net charge of the complex is positive and it is this positive charge that is believed to be important in facilitating DNA complex interaction with the negatively-charged cell membrane. Conversely, we now show that exactly the opposite is needed for efficiently delivering DNA/polycation complexes to skeletal muscle tissue in vivo.

For this study a variety of DNA/polycation complexes were formulated using three different charge ratios such that the net charge of complexes was either negative (two formulations) or positive (one formulation). Polycations used in this study included; proteins, polymers, lipids, polyamines, and combinations of each. In all cases the negatively charged complexes resulted in much higher levels of gene expression in rat muscle following delivery than the positively charged complexes (see table 6).

TABLE 6
Luciferase expression in rat leg muscles
following injection into the iliac artery of
negative vs. positive charge DNA complexes.

| | | Total nanograms of |
|---|---|---|
| | Charge Ratio | luciferase expression |
| | (negative to | per limb (all muscle |
| DNA/Polycation Complex | positive) | groups combined) |
| Polynucleotide alone | 1:0 | 1369.9 |
| DNA/cationic protein | 1:0.25 | 1908.8 |
| (histone H1) | 1:0.5 | 135.2 |
| | 1:2 | 69.1 |
| DNA/cationic polymer | 1:0.25 | 2355.3 |
| (linear PEI) | 1:0.5 | 1677.9 |
| | 1:2 | 7.2 |
| DNA/cationic protein + | 1:0.25 | 1551.1 |
| polyamine | 1:0.5 | 1181.9 |
| (histone H1 + polyamine 58) | 1:2 | 16.6 |
| DNA/cationic lipid | 1:0.25 | 537.3 |
| (DOTAP) | 1:0.5 | 171.6 |
| | 1:2 | 1.8 |
| DNA/cationic protein + | 1:0.25 | 863.4 |
| cationic lipid | 1:0.5 | 286 |
| (histone H1 + DOTAP) | 1:2 | 7.5 |

Conclusions: When using the bloodstream for gene delivery to skeletal muscle cells, the net charge of the complex is very important. Regardless of the type of polycation used for complexation with the DNA, net negatively charged complexes are much more efficient for gene delivery and expression than positively charged complexes.

2. Polynucleotide Delivery to Limb Skeletal Muscle Cells in Monkeys

A. Reporter Gene Systems

The pCI-Luc⁺ (Promega, Madison, Wis.) and pCI-LacZ plasmids express a cytoplasmic luciferase and the Escherichia coli LacZ, respectively, from the human cytomegalovirus (CMV) immediate-early promoter. The pCI vector (Promega) also contains an SV40 polyadenylation signal. pMI-Luc⁺ was constructed by replacing the CMV promoter in pCI-Luc⁺ with a 3300-bp murine muscle creatine kinase promoter. The vector pEBFP-N1 expresses a nuclear-localizing, blue-shifted green fluorescent protein (GFP) from the CMV promoter (Clontech, Palo Alto, Calif).

Luciferase assays were performed on muscle biopsies, entire muscles and various tissues as previously reported. The relative light units (RLU) were converted to nanograms of luciferase by using luciferase standards (Molecular Probes, Eugene, Oreg.) and a standard curve in which luciferase protein (pg)=RLU×5.1×10⁻⁵.

For the β-galactosidase assays, muscle samples were taken from the proximal, middle, and distal positions of each muscle, cut into small pieces, frozen in cold isopentane, and stored at −80° C. Muscle pieces were randomly chosen from each muscle sample (for every position) and 10 μm-thick cryostat sections were made. Every tenth section, for a total of 20 sections, was stained and analyzed. The sections were incubated in X-gal staining solution (5 mM potassium ferricyanide, 5 mM potassium ferrocyanide, 1 mM magnesium chloride, I mM X-gal in 0.1 M PBS , pH 7.6) for 4-8 h at RT and counterstained with hematoxylin and eosin. Three sections were selected randomly from the 20 sections of each position (usually the 4th, 11th and 17th sections, but an adjacent section was used if these sections were not intact). As previously described, the number of β-galactosidase-positive and total cells were determined within a cross area in each section by moving the counter grid from the top edge of the section to the bottom and from the left edge to the right. The percentage of β-galactosidase-positive cells for each muscle was gotten from the result of positive number divided by total cell number. A weighted average for the percent of transfected cells for each extremity muscle was determined as follows: (ΣAi*Mi)/M where Ai is percent of transfected cells for one muscle, Mi—weight of that muscle and M—whole weight of all muscles.

For the co-localization of β-galactosidase and GFP expression, 10 μm-thick cryostat sections were fixed with 4% formaldehyde for 5-10 min. Mouse-anti-β-galactosidase antibody and TRITC-labeled goat-anti-mouse IgG (Sigma) were used as primary and secondary antibodies, respectively. Using a Nikon Optiphot epifluorescence microscope with a SenSys CCD Camera (Photometrics, Tucson, Ariz.), two images were collected from the same view for TRITC-labeled β-galactosidase and for GFP and merged together using the program Adobe Photoshop 4.0.

B. Protocol

Seven Rhesus macaque monkeys (5 males; 2 females) of 6 to 13.7 kg body weight underwent intra-arterial injections in their limbs following anesthesia with ketamine and halothane. For the forearm injections, a longitudinal incision, ˜3 cm in length, was made on the skin along the inside edge of the biceps brachii and 2 cm above the elbow. After separating the artery from surrounding tissues and veins, a 20 g catheter was inserted into the brachial artery anterogradely and ligated in place. For the lower leg injections, the procedure was essentially the same as that used in the arm, but the incision was located on the upper edge of the popliteal fossae and the 20 g catheter was inserted into the popliteal artery.

For both the arm and leg injections, blood flow was impeded by a sphygmomanometer cuff surrounding the arm or leg proximal to the injection site. After the sphygmomanometer was inflated to more than 300 mmHg air pressure, the catheterized vessels were injected with 30 ml of normal saline containing 5 mg papaverine (Sigma Co.). Five min. later, a saline solution containing 100 μg pDNA/ml solution was rapidly injected within 30 to 45 sec. For the arms, the volume of each injection was 75 ml and 90 ml in the first two animals and 120 ml thereafter. The injection volume was ˜180 ml for the lower legs. The DNA solutions were injected using a nitrogen-pressurized cylinder. Two min after injection, the catheters were removed and the sphygmomanometer deflated.

The procedure was initially done on four monkeys in which one arm and leg was injected and muscle biopsies were taken at one (#1-3) or two weeks (#4). Monkey #2 had to be sacrificed at two weeks after injection because of an eye infection (unrelated to our procedure). Three more monkeys (#5-7) received an injection in all four extremities (one arm and leg on one day and the other two extremities two days later). Muscle biopsies were obtained at one week and the animals were sacrificed at two weeks after the injections. In monkeys #6 and #7, an arm and leg were injected with pCI-LacZ; all other injections were with pCI-Luc⁺.

C. Results:

All seven monkeys tolerated the procedure well and had full function of their arms, hands, legs and feet following the procedure. In particular, there was no indication of damage to the radial nerve, which could have been sensitive to the inflated sphygmomanometer surrounding the upper arm. Swelling in the target limbs, a putative correlate of successful gene transfer, was noted afterwards but completely subsided within one day. When the monkeys awakened from the anesthesia, 15 to 30 min after the procedure, they did not appear to be in any discomfort beyond that of normal surgical recovery. Occasionally, the skin in the target limb had some spots of hemorrhage which resolved within several days.

Four of the monkeys were sacrificed at 14 to 16 days after injection and all the target muscles of their limbs were assayed for either luciferase or β-galactosidase expression (Table 1). These results indicate that the intra-arterial injection of pCI-Luc⁺ DNA yielded levels of luciferase expression in all muscles of forearm, hand, lower leg and foot, ranging from 345 to 7332 ng/g muscle (Table 7). The variability in luciferase expression in arm muscles for different animals appears dependent upon whether the tip of the catheter was positioned in the radial or ulnar artery. The average luciferase expression levels in the limb muscles were 991.5±187 ng/g for the arm and 1186±673 ng/g for the leg.

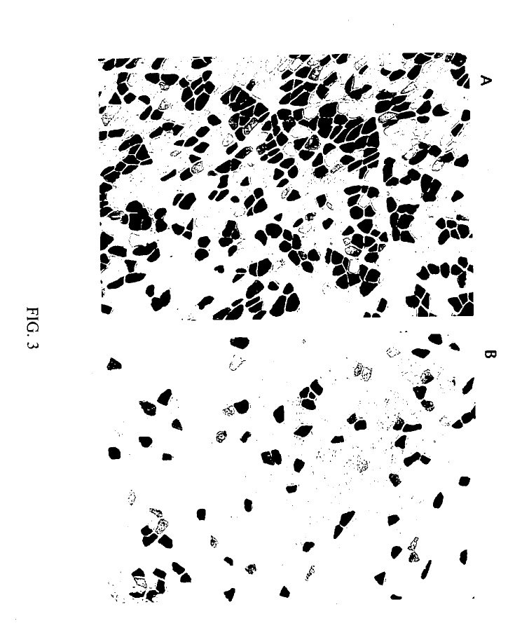

After intra-arterial injection of pCI-LacZ DNA, β-galactosidase expression was found in myofibers. Large numbers of β-galactosidase-positive myofibers were found in both leg and arm muscles, ranging from less than 1% to more than 30% in different muscles (Table 7 and FIG. 3). The average percentage for all four limbs injected was 7.4%, ranging from 6.3% to 9.9% for each of the limbs. The β-galactosidase percentages for specific muscle groups positively correlated with the luciferase levels in the same muscles (r=0.79).

TABLE 7
Mean muscle β-galactosidase or luciferase
expression in four muscles from monkeys sacrificed
two weeks after injection of pCI-LacZ or pCI-Luc⁺.
“±” indicates standard error;
n indicates the number of limbs assayed.

| | | | β-galactosidase | Luciferase |
|---|---|---|---|---|
| | | | (% positive) | (ng/g muscle) |
| Muscle group | | Muscle name | (n = 2) | (n = 5) |
| Arm muscles | | | | |
| Anterior | Superficial | palmaris longus | 5.9 ± 0.9 | 2368 ± 1309 |
| group | group | pronator teres | 19.9 ± 9.4 | 1818 ± 336 |
| | | flexor carpi radialis | 7.8 ± 0.7 | 1885 ± 762 |
| | | flexor carpi ulnaris | 3.8 ± 3.0 | 852 ± 314 |
| | | flexor digitorum spf. | 7.7 ± 1.2 | 1009 ± 189 |
| | Deep | flexor digitorum prof. | 1.0 ± 0.5 | 544 ± 360 |
| | group | pronator quadratus | 14.3 ± 11.1 | 1884 ± 331 |
| Posterior | Superficial | brachioradialis | 9.0 ± 8.7 | 1148 ± 942 |
| group | group | extensor carpi radialis longus | 6.6 ± 6.3 | 1179 ± 584 |
| | | extensor carpi radialis brevis | 9.4 ± 4.5 | 1118 ± 325 |
| | | extensor digitorum | 6.2 ± 5.4 | 1184 ± 94 |
| | | anconeus | 2.0 ± 0.3 | 1744 ± 372 |
| | | extensor carpi ulnaris | 0.6 ± 0.4 | 371 ± 86 |
| | | extensor pollicis longus | 6.9 ± 4.3 | 927 ± 228 |
| | Deep | supinator | 15.1 ± 9.3 | 2398 ± 748 |
| | group | abductor pollicis longus | 6.2 ± 3.8 | 927 ± 228 |
| | | extensor digiti secund et teriti | 6.0 ± 5.5 | 642 ± 168 |
| | | extensor digiti quart et minimi | 4.0 ± 3.5 | 593 ± 140 |
| Muscles | | muscle of thumb | 15.7 ± 0.5 | 904 ± 494 |
| of hand | | interosseus | 17.3 ± 4.3 | 1974 ± 185 |
| Weighted | | | 6.3 ± 0.04 | 991 ± 187 |
| Average | | | | |
| | | | β-galactosidase | Luciferase |
| | | | (% positive) | (ng/g muscle) |
| Muscle group | | Muscle name | (n = 2) | (n = 2) |
| Leg muscles | | | | |
| Posterior | Superficial | gastrocnemius | 3.0 ± 2.5 | 743 ± 33 |
| group | group | soleus | 21.2 ± 1.4 | 2888 ± 2151 |
| | Deep | popliteus | 37.1 ± 0.5 | 4423 ± 2657 |
| | Group | flexor digitorum longus | 8.9 ± 2.4 | 3504 ± 2151 |
| | | flexor hallucis longus | 9.7 ± 2.4 | 1355 ± 1224 |
| | | tibialis posterior | 28.7 ± 4.3 | 7332 ± 5117 |
| Anterior | | tibialis anterior | 2.8 ± 0.2 | 716 ± 162 |
| group | | extensor hallucis longus | 4.2 ± 1.4 | 810 ± 497 |
| | | extensor digitorum longus | 10.9 ± 1.0 | 3187 ± 1166 |
| | | abductor hallucis longus | 2.2 ± 0.2 | 345 ± 104 |
| Internal | | peronaus longus | 6.3 ± 2.5 | 626 ± 383 |
| group | | peronaus brevis | 8.9 ± 1.3 | 1300 ± 23 |
| Muscles of | | extensor digitorum brevis | 6.2 ± 5.0 | 672 ± 607 |
| foot | | extensor hallucis brevis | 2.4 ± 1.8 | 672 ± 607 |
| LEG MUSCLES | | | 7.3 ± 0.1 | 1692 ± 768 |
| Weighted | | | | |
| Average | | | | |

D. Delivery of Polynucleotide to the Diaphragm.

The monkey was anesthetized with ketamine followed by halothane inhalation. A 2 cm long incision was made in the upper thigh close to the inguinal ligament just in front of the femoral artery. Two clamps were placed around the femoral vein after separating the femoral vein from surrounding tissue. At an upstream location, the femoral vein was ligated by the clamp and a guide tube was inserted into the femoral vein anterogradely. A French 5 balloon catheter (D 1.66 mm) with guide wire was inserted into the inferior vena cava through the guide tube and an X-ray monitor was used for instructing the direction of guide wire. The guide wire was directed into the inferior phrenic vein. The catheter position in the inferior phrenic vein was checked by injecting iodine. The balloon was inflated to block blood flow through the inferior phrenic vein. 20 ml 0.017% papaverine in normal saline was injected. 5 minutes after papaverine injection, 40 ml of DNA solution (3 mg) was injected under elevated pressure (65 sec injection time). 2 minutes after DNA injection, the balloon was released and the catheter was removed. The animal was sacrificed and the diaphragm was taken for luciferase assay 7 days after the procedure.

TABLE 8
Luciferase expression in diaphragm from monkey
sacrificed 7 days after injection of pCI-Luc⁺.

| | total luciferase | ng luciferase / |
|---|---|---|
| diaphragm section | (ng) | gram if tissue |
| anterior part of left side | 0 | 0 |
| posterior part of left side | 0 | 0 |
| left conjunction area | 0 | 0 |
| anterior part of right side | 221.94 | 27.88 |
| posterior part of right side | 15.98 | 2.12 |
| right conjunction area | 34.21 | 17.82 |

E. Toxicity

We have administered ˜150 ml of fluid to ˜10 kg animals. While it was possible that this large amount of fluid might adversely affect the animal's cardiovascular or hemodynamic status, no adverse effects on the animals were observed.

Serum chemical and histological analyses were performed to determine if the procedure caused any adverse effects in the monkeys. The serum levels of creatine phosphate kinase (CK), alanine aminotransferase, aspartate aminotransferase (AST) and lactate dehydrogenase (LDH) after surgery were several times higher than before surgery. Levels peaked at 48 hours post-injection and returning to normal within several days. Other serum enzymes such as γ-glutamyltransferase (GGT) and alkaline phosphatase, hematological assays (hematocrit and RBC indices, platelets), serum electrolytes (Na, Cl, K), serum minerals (calcium, phosphate, iron), serum proteins (albumin, total protein), serum lipids (cholesterol, triglycerides), renal indices (urea, creatinine), and bilirubin were unaffected. Total WBC increased within the typical range post-surgery.

Limb muscles were obtained 14 to 16 days after intra-arterial injection and examined histologically. The vast majority of muscle tissue was well preserved and did not show any sign of pathology. In a few sections, mononuclear cells were noted surrounding β-galactosidase positive myofibers, some of which were undergoing degeneration. Immunostaining for CD-markers indicated that the majority of infiltrating cells were CD3-positive (T lymphocytes) with only a few B cells.

3. Model Systems:

A. Delivery of Polynucleotides to Limb Skeletal Muscle in Mdx Mice:

ICR or mdx mice, ˜30 gram, were anesthetized by intramuscular injection of ketamine(80-100 mg/kg) and xylazine (2 mg/kg). Metofane was added through inhalation if necessary during the procedure. A median incision was made from the upper third of abdomen to the caudal edge of the abdominal and the right caudal part of abdominal cavity was exposed using retractors. The tissue in front of the right external iliac artery was cleaned by forceps and a cotton tipped applicator. The arteries and veins to be clamped were separated from surrounding tissue and the caudal epigratric artery and vein, internal iliac arteries and vein, gluteal artery and vein, the vessels of deferent duct and external iliac artery and vein were clamped. A 0.6 ml of papaverine solution (containing 0.1 mg of papaverine) was injected into external iliac artery distal to the clamp. 2.5-3 ml of DNA solution containing 100 μg plasmid DNA was injected into the external iliac artery distal to the clamp with pressure 5 minutes post papaverine injection. A piece of gelfoam was put on the injection site before withdrawal of the needle and pressure was kept on the gelfoam to prevent bleeding. The clamps are taken off 2 minutes after injection and the abdominal cavity was closed by suturing.

Muscle samples were taken 7-10 days after injection and 6 μm thickness cryostat sections were made. Endogenous peroxidase activities were blocked by incubating the sections in 0.3% hydrogen peroxide in PBS for 5-10 min after the sections were mounted on slides and dried. The sections were rinsed twice with PBS (2 min×2 ) followed by Avidin/Biotin blocking by using Vector Avidin/Biotin Blocking Kit (Cat. No. SP-2001). The following steps were done according to the procedure of Vector M.O.M Immunodetection Kit (Cat. No. PK-2200). The immunofluorescent staining for human dystrophin in mouse muscle was done following the procedure of Vector M.O.M Immunodetection Kit (Cat. No. FMK-2201). For luciferase assays, 5 groups of muscle were taken from the whole injected leg according to their distribution, the anterior, posterior, medial, anterior of low leg and posterior of low leg. 2 ml of cold lysis buffer was added to each group of muscle followed by homogenization. Luciferase activity was measured by luminometer and the light units were converted to luciferase protein by using the converting rate of pg=light units×solution volume/20×2.05/100000

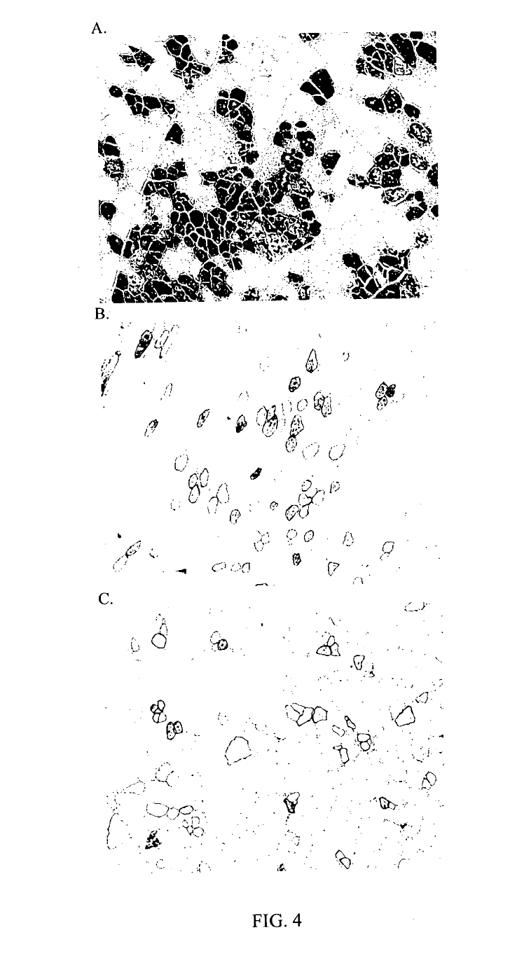

Results are shown in Table 9 and FIG. 4. FIG. 4A shows β-galactosidase expression in mdx dystrophic mouse. FIGS. 4B and C show human dystrophin expression in leg skeletal muscle in mdx and normal mouse, respectively.

TABLE 9
Luciferase expression in Mdx mouse.

| | total expression of whole leg | expression of per gram muscle |
|---|---|---|
| animal # | (ng luciferase) | (ng luciferase / gram muscle) |
| 134 | 1691 | 1271 |
| 135 | 1738 | 1307 |
| 137 | 1248 | 1177 |
| 138 | 869 | 643 |
| 140 | 1641 | 1357 |
| 141 | 881 | 663 |
| average | 1345 | 1070 |

B. Delivery of Polynucleotides to Limb Skeletal Muscles in Dystrophic Dog Model:

Juvenile male Golden Retriever dogs of 3 to 12 kg body weight underwent intra-arterial injections in their limbs following anesthesia. Anesthesia was with intravascular injection of propofol followed by isoflurane inhalant. For forearm injections, the arm was put at the extension and external rotation position and a 3 cm incision was made at the conjunction of armpit and upper arm and near the inside edge of the brachial biceps. After separating the brachial artery from the brachial vein and median nerve, a catheter (3-4F) was inserted anterograde into the brachial artery until the tip of the catheter reached to the elbow and was fixed by ligation. In some cases the brachial vein was clamped. Blood circulation of the forelimb was further inhibited by using a tourniquet placed around the upper limb up to the elbow (10 minutes maximum). For whole hindleg injections, an incision was made through the midline of the abdomen one inch below the umbilicus to the pubis. Connective tissue was separated to expose the common iliac artery and vein, external iliac artery and vein, internal iliac artery and vein, inferior epigastric artery and vein, superficial epigastric artery and vein, and the superficial iliac circumflex artery and vein. Clamps were placed on the inferior epigastric artery and vein, superficial epigastric artery and vein, and the superficial iliac circumflex artery and vein. An catheter (F5) was placed into the distal part of the iliac artery to the femoral artery and secured by ligation at the beginning of the femoral artery. Clamps are then placed on the external iliac vein, internal iliac artery and vein, and the common iliac artery and vein.

A 17% papaverine/saline solution was injected to increase vessel dilation (10-50 ml depending on animal size). After 5 minutes a plasmid DNA/saline solution was injected at moderately increased pressure using a nitrogen-pressurized cylinder set at 65 psi. For the forelimbs, the injection volume was 50-200 ml. For whole leg injections, the injection volume was 60-500 ml. Injection rates varied from 20 s to 120 s. Two min after injection, the clamps and tourniquet were released and the catheters were removed.

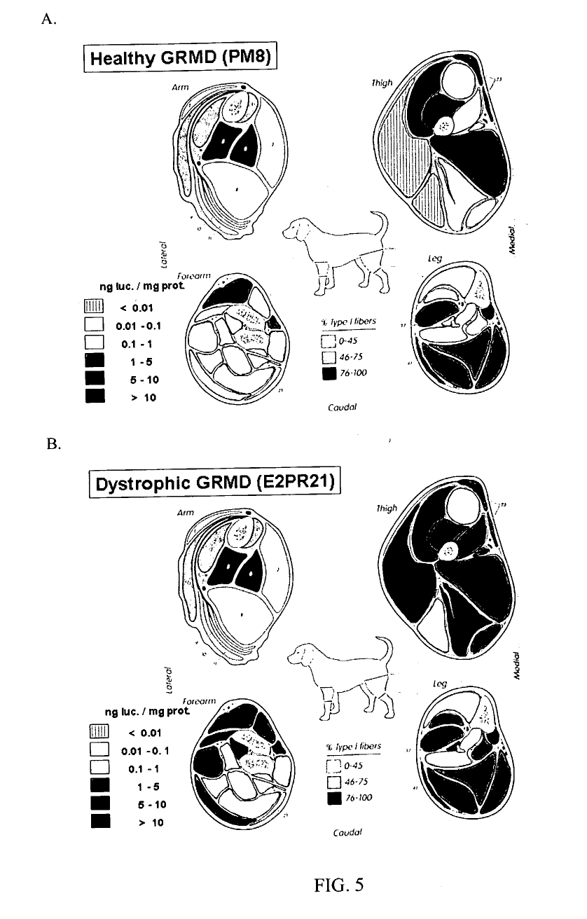

One forelimb and the opposite hindlimb or all four limbs were injected on day one with pMI-Luc+ (20-50 mg) or the dystrophin plasmid (50-330 mg). In these vectors, the reporter genes are under transcriptional control of the muscle creatine kinase promoter, which has been shown to direct sustained, high level expression in muscle. The animals were sacrificed at 7 days and all muscles were analyzed for gene expression. Uninjected limbs or limbs injected with saline were used to test for revertants. Results are shown in Table 10 and graphically summarized in FIG. 5. FIG. 5A illustrates the distribution of luciferase expression in normal dog. FIG. 5B illustrates the distribution of luciferase expression in the dystrophic dog model.

TABLE 10
Luciferase expression after of delivery pCI-Luc
polynucleotide in dog skeletal muscle cells.
Numbers given in pg Luciferase per mg total protein.

| | | GRMD dog | | healthy dog | |
|---|---|---|---|---|---|
| | | left | right | left | right |
| antebrachial muscles | | | | | |
| dorsolateral | extensor carpi radialis | | | 0.8 | 633 |
| | extensor digitorium | 5 | 1570 | | 299 |
| | communis | | | | |
| | extensor digitorium lateralis | | 7915 | | 438.5 |
| | extensor carpi ulnaris | | 671 | | 21.5 |
| | extensor pollicis longus | | 6763 | | 2456.7 |
| | et indicis proprius | | | | |
| | abductor pollicis longus | | 16724 | | 292.4 |
| | supinator | 9 | 14395 | 3.3 | 1920.8 |
| caudal | flexor carpi radialis | 3 | 828 | 1.5 | 116.2 |
| | flexor carpi ulnaris | | 270 | | 6.1 |
| | flexor digitorum | | 2017 | | 43.5 |
| | superficialis | | | | |
| | flexor digitorum profundus | | 49 | | 11.3 |
| | pronator teres | | 9231 | 5.2 | 270.6 |
| forepaw | forepaw | 10 | 958 | 2 | 1048.7 |
| other | brachi radialis | | | | 545.1 |
| muscles of the crus | | | | | |
| craniolateral | tibialis cranialis | 980 | 1.4 | 1.7 | |
| | extensor digitorum longus | 992 | 0.3 | | |
| | peroneus longus | 4116 | 0.3 | 127.8 | |
| | peroneus brevis | | | 6.2 | |
| | extensor digitorum lateralis | | 0.2 | | |
| caudal | gastrocnemius | 4365 | 0.1 | 3 | 0.1 |
| | flexor digitorum profundus | 1912 | 1.9 | 3 | |
| | tibialis caudalis | | 0.4 | | |
| | popliteus | 9821 | 0.3 | | |
| other | Testes | 0.1 | | | |
| | Liver #1 | | 0.3 | | |
| muscles of the pelvic limb | | | | | |
| thigh | gluteus superficialis | | 1.4 | 4.9 | |
| | gluteus medius | 4 | 0.2 | 0.1 | |
| | sartorius | | | 661.2 | |
| | tensor fasciae latae | | 0.5 | 369.7 | |
| | biceps femoris | 10312 | 1.1 | 0.1 | |
| | | | | 0.6 | |
| | semimembranosus | 5988 | 1.7 | 49.8 | |
| | semitendinosus | 432 | 1.1 | 0.1 | |
| | abductor magnus brevis | 4103 | 2 | 3644.8 | |
| | sartorius cranial part | 4664 | 0.9 | | |
| | rectus femoris | 396 | 0.1 | 179.9 | |
| | vastus medialis | 2588 | 0.5 | 7.4 | |
| | vastus intermedius | 4469 | 3.2 | 12448.7 | |
| | vastus lateralis | 2102 | 1 | 2927.8 | |
| | pectineus | 737 | 0.1 | 11.9 | |
| | gracilis | 1826 | 0.5 | 146 | |
| gluteal region | piriformis | 14 | 1.2 | | |
| and | gemellus | | 3 | | |
| hip joint | quadratus femoris | 911 | 0.1 | 1 | |
| | gluteus profundus | | 0 | 1.8 | |
| | PR21?obturator externus | | | 1.8 | |
| | biceps brachialis | | | | 0.1 |

The foregoing is considered as illustrative only of the principles of the invention. Furthermore, since numerous modifications and changes will readily occur to those skilled in the art, it is not desired to limit the invention to the exact construction and operation shown and described. Therefore, all suitable modifications and equivalents fall within the scope of the invention.

We claim:

1. An in vivo process for delivering a polynucleotide to dystrophic striated muscle cells, comprising:
a) inserting the polynucleotide into a blood vessel;
b) elevating blood pressure within a target tissue by controlling blood flow into and out of the target tissue; and,
c) delivering the polynucleotide to the striated muscle cells.

2. The process of claim 1 wherein the polynucleotide is in a complex having a zeta potential that is not negative.

3. The process of claim 1 wherein the polynucleotide is in a complex having a zeta potential that is not positive.

4. The process of claim 1 wherein the polynucleotide comprises a sequence whose presence in the striated muscle cell alters the endogenous properties of the striated muscle cell.

5. The process of claim 4 wherein the polynucleotide is an anti-sense polynucleotide.

6. The process of claim 4 wherein the polynucleotide is siRNA.

7. The process of claim 1 wherein controlling blood flow consists of externally applying pressure to blood vessels.

8. The process of claim 7 wherein externally applying pressure consists of compressing mammalian skin.

9. The process of claim 8 wherein compressing mammalian skin consists of applying a tourniquet over the skin.

10. The process of claim 8 wherein compressing mammalian skin consists of applying a cuff over the skin.

11. The process of claim 8 wherein compressing mammalian skin consists of applying a sphygmomanometer cuff over the skin.

12. A device for in vivo delivery of a polynucleotide to muscle cells, comprising: a cuff used over the skin to impede blood flow, thereby increasing delivery of the polynucleotide to muscle cells.

13. The process in claim 1 wherein the polynucleotide encodes all or a portion of the dystrophin gene and is delivered to striated muscle cells for the treatment of Duchenne or Becker Muscular Dystrophy.

14. The process of claim 1 wherein delivery is primarily to limb skeletal muscle cells.

15. An in vivo process for delivering a polynucleotide to striated muscle cells affected by Muscular Dystrophy, comprising:
a) forming a complex in solution consisting of the polynucleotide and an effective amount of a compound selected from the group consisting of polycations and cationic amphipathic molecules;
b) inserting the polynucleotide into a blood vessel;
c) elevating blood pressure within the target tissue by controlling blood flow into and out of a target tissue;
d) delivering the polynucleotide to the muscle cells; and,
e) maintaining full function of the tissue subsequent to the procedure.

16. The process of claim 15 wherein the amphipathic molecule consists of a lipid.

17. The process of claim 15 wherein the complex has a net charge that is less negative than the polynucleotide.

18. Process of claim 15 wherein the complex has a zeta potential that is not negative.

19. The process of claim 15 wherein the polycation is linked to the polynucleotide.

20. The process of claim 15 wherein a polyanion is added to the solution in sufficient amount to form a recharged complex.

21. The process of claim 20 wherein the recharged complex has a zeta potential that is not positive.

22. The process of claim 20 wherein the polyanion is linked to the polycation.
